(19) United States
(12) Patent Application Publication
(10) Pub. No.: US 2019/0358090 A1
(43) Pub. Date: Nov. 28, 2019

(54) FILM APPLICATOR ASSEMBLY

(71) Applicant: LABORATOIRE HRA-PHARMA, Châtillon (FR)

(72) Inventors: Eric AUGE, Paris (FR); Sabine MELINSTOCK, Paris (FR); Gladys CROSIER, Paris (FR); Paul OLIVES, Paris (FR); Gilles GARNIER, Paris (FR); Serge PARISY, Paris (FR); Nathalie GORGERY, Paris (FR); Sébastien RAHIR, Paris (FR); Cédric JEANMAIRE, Paris (FR); Jacky ROUSSEAU, Paris (FR)

(21) Appl. No.: 16/472,338

(22) PCT Filed: Dec. 21, 2017
(86) PCT No.: PCT/EP2017/084255

§ 371 Date: Jun. 21, 2019

Related U.S. Application Data
(60) Provisional application No. 62/437,660, filed on Dec. 22, 2016.

(51) Int. Cl. A61F 13/00; A61M 35/00; A61F 13/02

(52) U.S. Cl. CPC A61F 13/00085; A61M 35/003; A61M 2210/0625; A61F 13/00063; A61F 13/0259; A61F 13/0246

(57) ABSTRACT

The present invention relates to a film applicator assembly comprising a film layer and an applicator component for use on skin or mucosal surfaces.

### FIELD OF THE INVENTION

The present invention relates to film applicator assembly comprising a film layer and an applicator component for use on skin or mucosal surfaces.

### BACKGROUND OF THE INVENTION

Adhesive articles such as films and tapes are well known in the art and are used for various medical applications for humans and other mammals and for wound protection. Generally, the adhesive film or tape, itself, possesses adhesive properties or is coated with a pressure sensitive adhesive for securing the adhesive film or tape to bare skin or mucosal surface adjacent to a wound.

Some such adhesive films or tapes are very flexible or difficult to handle without wrinkling or adhering to themselves. Thus, applicators or delivery devices are used to apply the film or tape to the user's skin or mucosal surfaces. Examples of such systems includes U.S. Pat. No. 5,520,629 to Heinecke et al, U.S. Pat. No. 6,129,929 to Wick and U.S. Pat. No. 7,880,051 to Madsen et al., each of which patents are herein incorporated by reference in their entirety.

Each of these wound closure systems contact a substantial portion of the adhesive film or tape, increasing the difficulty of transferring the film or tape from the applicator. Therefore, a need exists for a wound dressing applicator assembly which facilitates release of the adhesive film or tape from the applicator once the adhesive film or tape contacts the user's skin.

Without being limited by theory, the inventors of the present invention have found that the force needed to remove a film layer from the applicator component is minimized by:
i) providing areas of discontinuous contact within a surface area region 3 contacted by applicator 2 to create separate regions (e.g., regions 3a, 3b or 3c) on top surface 1′ contacted by applicator 2 (as opposed to having only one continuous region contacted by applicator 2);
ii) restricting releasable, adhesive contact between applicator and film layer to the peripheral surface area; and/or
iii) reducing the total surface area of any contact region(s) on top surface 1′ formed by contact with the applicator 2.

One aspect of the present invention, therefore, relates to a film applicator assembly comprising a film layer and an applicator which facilitates release of the film from the applicator once the adhesive film contacts and adheres to the user's skin.

Another aspect of the present invention relates to a film applicator assembly comprising a film layer having a surface having adhesive properties and an applicator, wherein the applicator releasably, adhesively contacts (continuously or discontinuously) the adhesive surface of the film layer along the periphery of the surface.

One other aspect of the present invention relates to a film applicator assembly comprising a film layer having a surface having adhesive properties and an applicator, wherein the applicator releasably adhesively contacts (continuously or discontinuously) the surface of the film layer such that the surface area of the surface of the film layer releasably adhesively contacting the applicator is less than about 40%, optionally less than 30%, optionally less 20%, optionally less than 10%, optionally less than 3% of the total surface area of the surface of the film layer, but greater than 1% of the total surface area of the surface of the film layer. Optionally, the surface area of the surface of the film layer releasably adhesively contacting the applicator is less than about 40% to about 1%, optionally from about 30% to about 2%, optionally, from about 20% to about 3%, optionally from about 10% to about 4%, or optionally, about 6% of the total surface area of the surface of the film layer

### SUMMARY OF THE INVENTION

In certain embodiments, the present invention relates to film applicator assemblies comprising:
a) a film layer having a top surface having adhesive properties and one or more outer edges, the top surface having a surface area; and
b) an applicator releasably adhesively connected to the top surface, contacting at least one contact region of the top surface of the film layer to which it is releasably affixed;
wherein the total surface area of the contact regions contacted by the applicator is less than about 40% of the total surface area of the top surface of the film layer to which the applicator is releasably adhesively connected.

In certain embodiments, the present invention relates to film applicator assemblies comprising:
a) a film layer having a top surface having adhesive properties, the top surface having a surface area; and
b) an applicator releasably adhesively connected to the top surface of the film layer, contacting at least two contact regions of the top surface of the film layer to which it is releasably affixed;
   wherein the total surface area of the contact regions contacted by the applicator is less than about 40% of the total surface area of the top surface of the film layer to which the applicator is releasably adhesively connected.

In certain embodiments, the present invention relates to film applicator assemblies comprising:
a) a film layer having a top surface having adhesive properties, the top surface having:
   i. an outer edge defining a perimeter
   ii. a surface area, the surface area having a peripheral surface area and an internal surface area, the peripheral surface area extending from the perimeter to the internal surface area; and
b) an applicator releasably adhesively connected to the top surface of the film layer at one or more contact regions within the peripheral surface area of the film layer wherein the contact regions do not extend within the internal surface area.

In certain embodiments, the present invention relates to film applicator assemblies comprising:
a) an applicator having a gripping end and an applicator end opposite the gripping end, the applicator end having a surface and a cutout formed therein; and
b) a film layer defining a top surface and one or more outer edges, the top surface having a surface area, the top surface of the film layer being in releasable adhesive contact with the surface of the applicator end and overlaying the cutout such that less than 40% of the surface area of the top surface of the film layer is in releasable adhesive contact with the applicator end.

### BRIEF DESCRIPTION OF THE DRAWINGS

An embodiment of this invention will now be described in greater detail, by way of illustration only, with reference to the accompanying drawings, in which:

FIG. 1A is a top view of a film layer having a circumferential edge of the present invention;

FIG. 1B is a top view of a film layer having triangular edges;

FIG. 1C is a top view of a film layer having longitudinal and transverse edges;

FIG. 2 is a top view of an embodiment of the film applicator assembly of the present invention showing attachment of applicator to film layer;

FIG. 3 is a bottom view of the film applicator assembly of FIG. 2 showing discontinuous attachment of applicator to film layer at contact regions;

FIG. 3A is a bottom view of the film layer of FIG. 3 showing contact regions as discontinuous.

FIG. 4 is a top view of an embodiment of the film applicator assembly of the present invention showing attachment of applicator to film layer;

FIG. 5 is a bottom view of the film applicator assembly of FIG. 4 showing continuous attachment of applicator to film layer at contact region;

FIG. 5A is a bottom view of the film layer of FIG. 5 showing contact region as continuous.

FIG. 6 is a top view of an embodiment of the film applicator assembly of the present invention showing attachment of applicator to film layer;

FIG. 7 is a bottom view of the film applicator assembly of FIG. 6 showing continuous attachment of applicator to film layer at contact region;

FIG. 7A is a bottom view of the film layer of FIG. 7 showing contact region as continuous;

FIG. 8 is a top view of the film applicator assembly of FIG. 2 showing release liner 8 covering top surface 1′ of the film layer 1;

FIG. 9 is an exploded view of components of film applicator assembly and wrapper packaging; and

FIG. 10 illustrates use of film applicator assembly to apply film layer to skin surface of lip of a user.

### DETAILED DESCRIPTION OF THE INVENTION

The film applicator assembly of the present invention can comprise, consist of, or consist essentially of the essential elements and limitations of the invention described herein, as well any of the additional or optional features, components, or limitations described herein.

The term “comprising” (and its grammatical variations) as used herein is used in the inclusive sense of (and, interchangeably with the terms) “having” or “including” and not in the exclusive sense of “consisting only of.” The terms “a” and “the” as used herein are understood to encompass the plural as well as the singular.

All documents incorporated herein by reference, by portion or in their entirety, are only incorporated herein to the extent that they are not inconsistent with this specification.

In certain embodiments, the present invention as disclosed herein may be practiced in the absence of any component, element (or group of components or elements) or method step which is not specifically disclosed herein.

As shown in FIGS. 2-3 and 4-5, the film applicator assembly 10 of the present invention comprises a film layer 1 having top surface 1′ and a bottom surface 1″, opposite the top surface 1′, the top surface 1′ having adhesive properties. In certain embodiments, the adhesive properties on the top surface 1′ define an adhering surface for application to anatomical surfaces of humans and/or animals (e.g., the skin or mucous surfaces) and releasably adhesively connecting the film layer 1 to an applicator 2 for transfer to such anatomical surfaces. As used herein, “adhesive property(ies)” with respect to a film surface means the ability of that film surface to act as an adhesive due to: i) properties of the film forming material (or formulation) used to form such film surface (e.g., the adhesive layer of film layer 1 described below); or ii) due the presence of a separate adhesive coating coated on the surface of top surface 1′ of film layer 1.

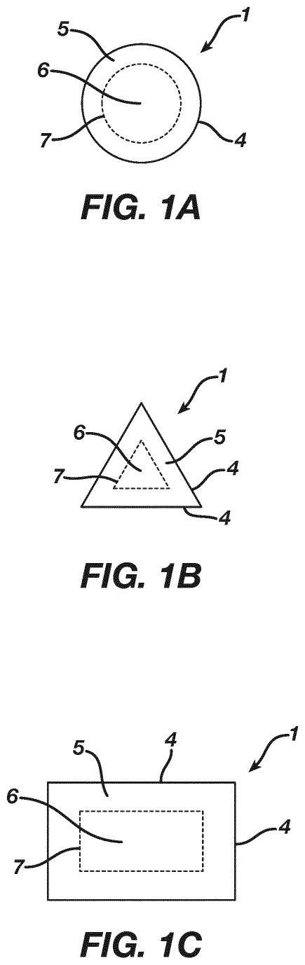

In an embodiment, the film layer 1 further comprises an outer edge(s) 4 which, or the total of which outer edges in the case of multisided planar geometric shapes, defines a perimeter (or outer point of the film layer 1 where periphery terminates). As illustrated in FIGS. 1A-C, the outer edge 4 comprises any edge of a planar geometrically shaped film layer, including a circumferential edge, a triangular edge, a longitudinal edge, a transverse edge, or any side edge of a multisided geometric shape. The top and bottom surfaces 1′ and 1″, each, have a surface area, the surface area having a peripheral surface area 5 and an internal surface area 6, the peripheral surface area 5 extending from the perimeter (or from a location adjacent to or near the perimeter) to the internal surface area 6. Peripheral surface area 5 and internal surface area 6 are illustrated in FIGS. 1A-C, and 2-5 as portions of the total surface area of a top or bottom surface (1′ or 1″) separated by imaginary dotted line 7.

The film applicator assembly 10 is typically constructed so that the film layer 1 is releasable affixed to an applicator 2, which applicator 2 used to transfer and facilitate application of the film layer 1 to a surface (e.g., as described above). In certain embodiments as illustrated in FIGS. 2-3, 4-6 and 7, the applicator 2 comprises an applicator end 17 having surface 17′, gripping end 18 opposite the applicator end 17, and cutout 15, the cutout 15 forming cutout periphery 16. In certain embodiments, the cutout 15 is positioned at the applicator end 17 so as to form an “open” cutout (i.e., a cutout which is not bound by a cutout periphery 16). Alternatively, the cutout 15 is positioned at the applicator end 17 so as to form an “closed” cutout (i.e., a cutout which is completely bound [or surrounded] by a cutout periphery 16). The applicator 2 is releasably adhesively connected to the film layer 1 so as to minimize the force necessary to remove the film layer 1 from the applicator 2. The adhesive properties of the top surface 1′ of film layer 1 are such that the top surface 1′ of film layer 1 remains in releasable adhesive contact with the applicator 2 upon removing (or after removal of) any optional release liner (such as release liner 8 [see below]) which may be used as protective liner protecting the adhesive side(s) of the film layer 1 from applicator 2

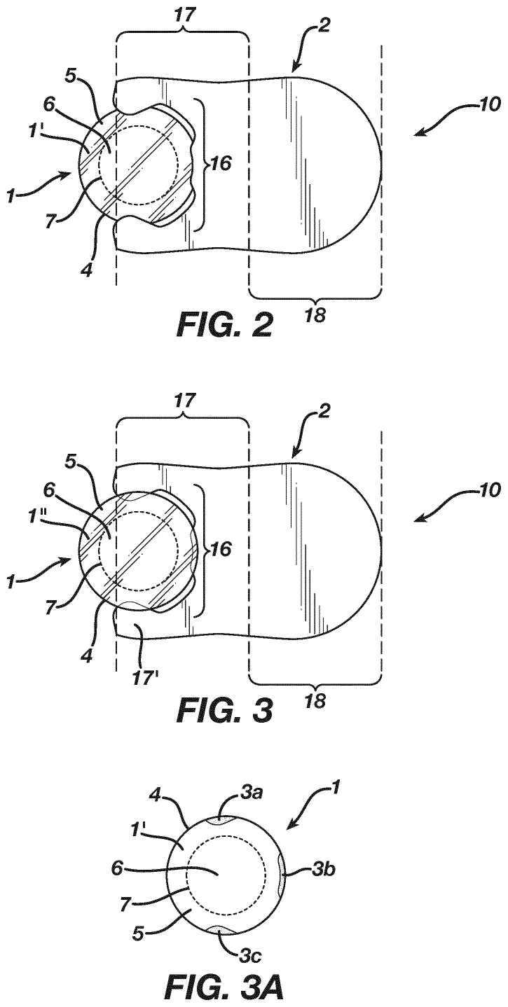

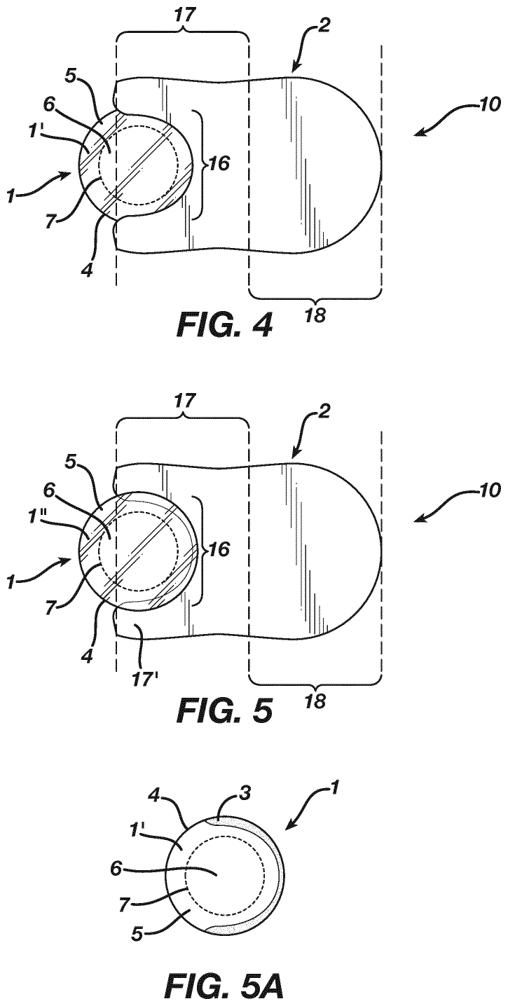

In another embodiment, as shown in FIGS. 3, 3A, 5 and 5A, the surface 17′ of applicator 2 is releasably adhesively connected to a top surface 1′ (and/or, optionally, bottom surface 1″) at continuous (FIGS. 4-5A) or discontinuous (FIGS. 2-3A) contact region(s) 3 within the peripheral surface area 5. The contact region 3 represents the region in which the applicator 2 releasably adhesively contacts the surface area of top surface 1′ (and/or, optionally, the bottom surface 1″) to achieve the releasable adhesive connection. The releasable adhesive connection results from adhesiveness of the top surface 1′ due to its adhesive properties. In certain embodiments, a separate adhesive may be provided across the contact region 3 if necessary to provide sufficient adhesive contact for maintaining connection of film layer 1 to applicator 2 prior to transfer of film layer 1 to user's skin or mucosal surface; if necessary, suitable such adhesives include any conventional adhesive known for such use, as for example pressure acrylic adhesives, among others. Additionally, such an adhesive may contain a resin for increasing adhesion, a cohesion increasing agent, an absorption agent (preferably a polyacrylate superabsorbent, a polyacrylate salt superabsorbent or a mixture thereof), a plasticizer and optionally a pigment. Such adhesives are also useful as the, optional, adhesive coating (not shown) for providing adhesive properties as defined above. In certain embodiments, the force required to overcome the adhesive force between the applicator 2 and the film layer 1 is less than the force required to overcome the adhesive force between the film layer 1 and the skin or mucosal surface of a human or animal. In certain embodiments, applicator 2 is releasably adhesively connected to the top surface 1′ within the peripheral surface area 5, yet not extending within the internal surface area 6.

In an embodiment, as illustrated by FIGS. 2-3A, the applicator 2 is releasably adhesively connected to film layer 1 at discontinuous (or separate, discrete) contact regions 3 of top surface 1′ of film layer 1 so as to reduce and/or prevent folding of film layer 1 onto itself, drag of film layer from surface of application or tearing of the film during application. Optionally, the applicator 2 is releasably adhesively connected to film layer 1 at least two discontinuous (or separate, discrete) contact regions 3 of top surface 1′ of film layer 1, or, optionally at at least three separate, discrete regions of top surface 1′ of film layer 1 as illustrated by distinct regions 3a, 3b, and 3c.

In another embodiment, as illustrated by FIGS. 4-5A, the applicator 2 is releasably adhesively connected to film layer 1 at a continuous contact region 3 of top surface 1′ and a contact a contact region 3 on the bottom surface 1″ of film layer 1.

In certain embodiments, the surface area contact region(s) 3 is less than about 40% to about 1%, optionally from about 30% to about 2%, optionally, from about 20% to about 3%, optionally from about 10% to about 4%, or optionally, about 6% of the total surface area on which the contact regions are located.

### Film Layer

Film layer 1 protects the wound site once, as described below, the film layer 1 is detached from applicator 2 and transferred to the skin or mucosal surface of the user, generally at site a wound or other site in need of barrier protection or moisture retention.

Film layer 1 is thin, highly flexible or deformable, water-impervious, or substantially impervious to bodily fluids, yet breathable. In some embodiments, film layer 1 is water-impervious, or substantially impervious to bodily fluids.

In certain embodiments, the film layer 1 is formed so as to be conformable to the contours of the body, and flexible so as to permit free movement of the body part wearing the film layer 1. Film layer 1 is very lightweight, and may be elastic (elastomeric) in character.

In certain embodiments, the wound cover provides a humid environment but without saturation, cicatrisation, which, without being limited by theory, is a situation suitable for acceleration of the healing.

In certain embodiments, film layer 1 of the present invention comprises an adhesive layer comprising a blend of at least three basic ingredients, viz., the hydrophobic unsaturated aliphatic homopolymer, a compatible tackifier, and at least one hydrocolloid absorbent. This mixture of ingredients is exposed to a dose of ionizing radiation which chemically cross-links the hydrophobic unsaturated aliphatic homopolymer component, thereby yielding a high integrity film layer 1. While it is preferable to irradiate the ingredients after mixing and formation into a desired shape (e.g. an adhesive sheet), it is possible to irradiate the ingredients prior to mixing and/or formation. However, in such an instance, the complete mixing of the ingredients may be impeded, and the resulting mixture may still need to exposed to a further dose of radiation to deliver the high integrity the adhesive layer of film layer 1.

The hydrophobic unsaturated aliphatic homopolymer can comprise either a straight-chain unsaturated aliphatic homopolymer, a branched unsaturated aliphatic homopolymer, or a combination thereof. In addition, the hydrophobic unsaturated aliphatic homopolymer can be substituted along its polymer chain with another moiety, such as chlorine, fluorine, or a lower alkyl, and still be considered to fall within the scope of the present invention. However, substitution of other monomers within the polymer chain of the homopolymer (e.g., random, block, and sequential copolymers) is not considered to be within the present invention.

As used herein, a hydrophobic unsaturated aliphatic homopolymer refers to organic homopolymers, typically olefin homopolymers, that are substantially water insoluble, and which exhibit a significant degree of unsaturated double bonds in the homopolymer chain and/or branched side chains. While potentially any degree of unsaturation may serve to form the film layer 1, the hydrophobic unsaturated aliphatic homopolymer preferably exhibits at least about fifty mole percent (50%) unsaturation, and more preferably at least about ninety mole percent (90%) unsaturation. In an especially preferred embodiment, the hydrophobic unsaturated aliphatic homopolymer exhibits virtually one hundred mole percent (100%) unsaturation, i.e. essentially 100% unsaturated double bonds per monomer unit of the homopolymer.

Preferably, the hydrophobic unsaturated aliphatic homopolymer comprises an elastomeric homopolymer. Nonlimiting examples of suitable elastomeric homopolymers include polyisoprene, polybutadiene, and combinations thereof, with polyisoprene being particularly preferred. Polyisoprene is commercially available from a number of sources, including Goodyear Chemical Co., Akron, Ohio, under the NATSYN™ trademark, including Natsyn resin Nos. 2200, 2205, and 2210.

The hydrophobic unsaturated aliphatic homopolymer preferably comprises from about 20 percent to about 50 percent by weight of total weight of the adhesive layer of film layer 1 of the present invention. For covering/protecting wounds or sores, it is desirable to limit the amount of hydrophobic unsaturated aliphatic homopolymer present, in order to maximize the level of hydrocolloid, thereby achieving maximum fluid absorbency. Thus, when forming films for covering/protecting wounds or sores, from about 25 weight percent to about 35 weight percent of the hydrophobic unsaturated aliphatic homopolymer is employed.

The compatible tackifier can comprise either an elastomeric tackifier, such as polyisobutylene, or a non-elastomeric tackifier, including synthetic polyterpene tackifiers, such as WINGTACK™ brand tackifiers (e.g., Wingtack 10, Wingtack 86, Wingtack 95, Wingtack Plus, and Wingtack Extra) available from Goodyear Chemical Co., Akron, Ohio, or a combination of elastomeric and non-elastomeric tackifiers. As used herein, a “compatible tackifier” refers to a tackifier that is miscible with the hydrophobic unsaturated aliphatic homopolymer, such that when these components are mixed they form a homogeneous phase.

Preferably, the compatible tackifier comprises low molecular weight polyisobutylene (viscosity average molecular weight of from about 20,000 to 70,000, preferably from about 40,000 to about 65,000). Suitable low molecular weight polyisobutylene tackifiers are available from Exxon Chemical Company under the tradenames Vistanex LM and Vistanex L-100, respectively, and include Vistanex LM-MS (viscosity average molecular weight=44,000), Vistanex LM-MH (viscosity average molecular weight=53,000), and Vistanex LM-H (viscosity average molecular weight=63,000).

The compatible tackifier preferably comprises from about 20 weight percent to about 60 weight percent, and more preferably from about 30 weight percent to about 50 weight percent of the film layer 1 of the present invention.

In certain embodiments, the hydrocolloid absorbent comprises synthetic polymers prepared from single or multiple monomers, naturally occurring hydrophilic polymers or chemically modified naturally occurring hydrophilic polymers. The hydrocolloid polymers may be linear or cross-linked. This include natural or chemically modified natural polymers like cellulosics such as CMC, chitosan, pectin, guar gum, starches or dextrines, collagenes and gelatine and synthetic polymers like polyacrylic acid, polyvinylealcohol/acetate, polyhydroxyalkyl acrylates and methacrylates, polyacrylamides, polystyrene sulfonates, polyvinyl pyrilidone, polyglycols, copolymers, grafts of such, copolymers or compositions of such.

In preferred embodiments, the hydrocolloid absorbent comprises a natural hydrocolloid, such as pectin, gelatin, or carboxymethylcellulose (CMC) (Aqualon Corp., Wilmington, Del.), a semi-synthetic hydrocolloid, such as cross-linked carboxymethylcellulose (X-link CMC) (e.g. Ac-Di-Sol; FMC Corp., Philadelphia, Pa.), a synthetic hydrocolloid, such as cross-linked polyacrylic acid (PAA) (e.g., CARBOPOL™ No. 974P; B.F. Goodrich, Brecksville, Ohio), or a combination thereof. Preferably, the hydrocolloid absorbent component comprises from about 5 percent to about 60 percent by weight of the adhesive layer of film layer 1. When preparing the adhesive layer of film layer 1 for use in protecting a wound or sore, the hydrocolloid absorbent preferably comprises from about 20 percent to about 40 percent by weight of the adhesive layer.

The particular selection of the hydrocolloid absorbents to be used in any one adhesive formulation will depend upon the intended use for that formulation. For example, in preparing the adhesive layer of film layer 1 for use in covering/protecting a wound or sore, maximum absorbency without a loss of wet integrity is desired. Thus, a major portion of the hydrocolloid absorbent should comprise natural hydrocolloids that are water soluble, and provide maximum absorbency. In addition, cross-linked semi-synthetic and synthetic hydrocolloids, which are water swellable, but water insoluble, may also be included in the formulation for the adhesive layer of the film layer 1 to serve as a filler, and/or to help regulate the swelling of the formulation for the adhesive layer of the film layer 1.

Thus, the absorbency of the adhesive layer of film layer 1 of the present invention can be adjusted based on the particular need. In general, when formulating an film layer 1 for use in a covering/protecting a wound or sore, the formulation for the adhesive layer of film layer 1 will preferably exhibit an absorbency of at least 50 percent, and more preferably an absorbency of from about 100 percent to about 500 percent after twenty-four hours of exposure to aqueous fluids.

The use of cross-linked polyacrylic acid (PAA) as a hydrocolloid absorbent may provide additional advantages to the adhesive layer of film layer 1. Specifically, the acidic nature of PAA lowers the overall acidity of the formulation for the adhesive layer of film layer 1 from a pH of about 7 to a pH of about 5. When such a formulation is employed in a covering/protecting a wound or sore, the pH of the wound exudate will likewise be lowered. This in turn may lead to promotion of more rapid healing of the wound or sore. See e.g., K. Tsukada et al., “The pH Changes of Pressure Ulcers Related to the Healing Process of Wounds”, 4, WOUNDS: A Compendium of Clinical Research and Practice, 16 (January-February, 1992).

The adhesive layer of film layer 1 of the present invention may also optionally contain a plasticizer component at from about 0.5 percent to about 10 percent by weight of the total adhesive layer of film layer 1. Preferably, the plasticizer comprises mineral oil (Spectrum Corp., Gardena, Calif.).

Compositions of the present invention may also contain minor amounts of other ingredients such as antioxidants, deodorants, perfumes, antimicrobials and other pharmacologically active agents as is well known in the art. Furthermore, additional elastomers, including polypropylene-polyethylene copolymers such as EPSYN™ resins available from Copolymer Rubber and Chemical Corp., Baton Rouge, La., can also be included in the compositions of the present invention.

Compositions of the invention are made by compounding the hydrophobic unsaturated aliphatic homopolymer and compatible tackifier with a heavy duty mixer until a homogeneous blend is obtained, forming an adhesive phase. Small portions of a dry-blended premix of one or more hydrocolloid absorbents are added and milling continued until a homogeneous dispersion of the absorbents in the adhesive phase is obtained. The blended adhesive mass is then molded into films such as adhesive layer incorporated into or used as film layer 1. In addition, the blended adhesive mass can also be fed into a heated single-or dual-screw extruder and coated from a standard extrusion die to form adhesive sheets capable of being converted into appropriately shaped materials.

After formation, the adhesive layer of film layer 1 of the present invention are irradiated with a dose of ionizing radiation at from about 5 kGy (0.5 Mrad) to about 200 kGy (20 Mrad), more preferably at a dose of from about 25 kGy (2.5 Mrad) to about 50 kGy (5 Mrad). Both E-beam and gamma irradiation can serve as the ionizing radiation source used to irradiate the adhesive layer of film layer 1, and thereby chemically cross-link the hydrophobic unsaturated aliphatic homopolymer component of the adhesive layer of film layer 1. It is this cross-linking of the hydrophobic unsaturated aliphatic homopolymer component that results in the consistently high wet integrity displayed by the adhesive layer of film layer 1. In addition, the application of ionizing radiation can also be used to sterilize the film layer 1.

The degree of cross-linking in the adhesive layer of film layer 1 of the present invention can be gauged by measuring the percent gel content of the hydrophobic unsaturated aliphatic homopolymer component after being exposed to a pre-determined dose of ionizing radiation. Specifically, the irradiated hydrophobic unsaturated aliphatic homopolymer is placed in a nonpolar organic solvent, such as hexane, heptane, or toluene, that is normally capable of dissolving the homopolymer. Any cross-linked homopolymer will form a gel in the solvent, while non-cross-linked homopolymer will dissolve. The remaining gelled homopolymer is removed from the solvent, washed, dried, weighed, and expressed as a percent by weight of the original irradiated material. This gel percent measurement can then be used to gauge the amount of cross-linking, and thereby the specific dose of radiation required to yield the high wet integrity compositions of the present invention. Accordingly, the hydrophobic unsaturated aliphatic homopolymer component of the compositions of the present invention should preferably exhibit at least 50 percent gel content, and more preferably at least 70 percent gel content. The specific dosage of radiation required to reach this level of gel content will depend upon the particular hydrophobic unsaturated aliphatic homopolymer chosen for inclusion in the compositions of the present invention.

In certain embodiments, the adhesive layer has a thickness in the range of 20-300 μm, such as 25-300 μm, such as 30-200 μm, such as 25-150 μm, such as 30-100 μm, and the vapour permeability of the dressing sheet being 50-1000 g/m², such as 100-800 g/m², such as 200-700 g/m², such as 350-650 g/m² measured over 24 hours using the “Water Method” of ASTM E 96-00 as detailed in “ASTM Standard Designation E 96-00”, published in July 2000, “Standard Test Methods for Water Vapor Transmission of Materials”.

The film layer 1 may preferably have an absorption of 50-400 g/m²/6 h, more preferred 60-300 g/m²/6 h and most preferred 70-250 g/m²/6 h. The absorption is determined by immersing the patch in 0.9 M saline water at 37° C. for 6 hours and then measuring the water uptake.

Very thin dressings or patches are usually prepared with non-absorbent adhesives, such as polyacrylates. The presence of hydrocolloid absorbent, however, provides a moist wound healing environment by absorbing moisture, thus leaving the wound or skin neither too dry nor too wet. The absence of absorbent particles may give rise to maceration of the skin or drying out of a wound.

The thickness of the adhesive layer of the film layer 1 of the present invention is substantially constant across the surface.

It is preferred that the thickness of the adhesive layer of film layer 1 is 20-300 μm, more preferred 25-150 μm and most preferred 30-100 μm and even most preferred 50-80 μm.

The film layer 1 has a substantially uniform thickness. Due to the low thickness, beveling may not be necessary in order to ensure good tack and reduce rolling up of the edge portions.

In certain embodiments, the film layer 1 has a total thickness of 100-200 μm. The obtained film layer 1 is thus thick enough to be handled without folding or wrinkling but at the same time remarkably thinner than traditional hydrocolloid dressings.

The surface area of the top surface 1′ of film layer 1 may e.g. be less than 5 cm², such as at most 4 cm², such as at most 2 cm², such as in the range of 1-2 cm², or smaller, such as 0.08-1 cm², such as 0.1-0.8 cm², such as 0.12-5 cm². For facial application, the surface area of top surface 1′ is usually less than 5 cm².

In certain embodiments, the hydrocolloid absorbent is in the form of particulates and the particle size can influence on the thickness of the adhesive layer, as it is difficult to prepare an adhesive layer being thinner than the size of the particles of the hydrocolloid absorbent.

The physical form of conventional hydrocolloids is relative coarse and irregular particles, typically about 60-100 μm and the particles are in the form of a dry powder. In order to obtain finer particles, the hydrocolloid may be milled and/or sifted.

In a preferred embodiment of the invention, the hydrocolloid absorbent particles have an average size being substantially less than 125 μm, more preferred less than 100 μm, even more preferred less than 75 μm and most preferred less than 50 μm.

The adhesive layer forms the top surface 1′ may have patterns formed therein, such as geometric pattern or a random pattern, or the top surface 1′ formed by the adhesive layer may comprise larger interruptions in the form of areas with no adhesive, e.g. the central part of the patch.

It is preferred that the top surface 1′ formed by adhesive layer is uninterrupted. The uninterrupted layer provides several advantages, such as less wrinkling, better invisibility and better blending into the skin.

The film layer 1 may be in a flat continuous layer from 20 g/m² up to 1000 g/m².

In certain embodiments, the film layer 1 further comprises a backing layer on the surface of the adhesive layer opposite top surface 1′. In certain embodiments, the backing layer is a substantially water-impervious film which protects the adhesive from being adversely affected when the wearer is bathing or in case of incidental wetting of the area and especially when the adhesive is water absorbing. However, the backing layer is also vapor permeable.

The backing layer may be any water impervious layer or film may be of any suitable material known per se for use in the preparation of wound dressings that also permits vapor permeability—e.g. a foam, a non-woven layer or a polyurethane, polyethylene, polyester or polyamide film. In accordance with the invention it has been found in practice that the use of a thinner backing layer or film than is normally used when preparing medical dressings, an improved stretchability and adaptability is obtained at the same time as the modulus is reduced.

The backing layer may preferably be an elastic, flexible and non-sticking film that protects the adhesive during storage as well as during use.

The water impervious, but vapor permeable, layer or film is preferably a low-friction flexible polymer film reducing the risk of unwanted stress in the area of application.

An especially suitable material for use as a water impervious film is a polyurethane film. A preferred low friction film material is disclosed in U.S. Pat. No. 5,643,187, herein incorporated by reference in its entirety.

If the film layer 1 is desired for an “invisible” face patch, a rather thin film would be appropriate, the backing layer has a thickness of less than 30 μm.

In certain embodiments, the thickness of the backing layer may be below 20 microns, more optionally from about 9 to about 18 microns, or optionally from about 9 to about 11 microns.

In certain embodiments, the backing layer may be opaque, with a reflection being near to the reflection of skin.

The backing layer may be coloured in suitable colours, e.g. flesh-colour or it may carry ornamentals. The backing layer may be transparent, translucent, opaque or non-transparent, depending on the intended use.

In order to visually blend into the skin and become invisible, it is desired that the film layer 1 have a reflectance being close to that of the skin. It is preferred that the reflectance is lower than 5, more preferred between 4.5 and 1, and even more preferred between 4 and 1.5. The reflectance is measured on a Micro-TRI-gloss apparatus from BYK-Gardner and is measured with reference to an ASTM D523 standard. The measuring angle is 60°. The higher value of reflectance, the higher is the gloss of the product.

In an embodiment, film layer 1 is substantially transparent. This may be particularly desirable to protect small wounds, such as cold sores or non-bleeding cuts or and the like, without visible bandages. In other embodiments, Film layer 1 is substantially translucent. In still other embodiments, Film layer 1 is substantially opaque. An opaque Film layer 1 can serve to hide the wound from view once film layer 1 is applied. In other embodiments, such as for use in children's film applicator assemblies 10, film layer 1 may be decorated. Decorations include color or colors, decals, printed messages, or cartoons. The decoration serves the dual purpose of hiding the wound site from view, as well as providing entertainment for the wearer of the film layer 1.

In general, the total thickness of film layer 1 (including any backing layer as described above) is between about 70 to about 110 microns (“μm”), optionally between about 85 μm to about 95 μm and optionally, about 90 μm to achieve the forming and flexing characteristics desired.

In certain embodiments, the film layer 1 of the invention is sterilized to avoid the risk of causing infections when applied to skin areas having broken skin.

It is not critical whether or not the film layer 1 is sterilized if the film layer 1 is applied to non-broken skin. However, if the film layer 1 is applied to broken skin, the film sterilization may be preferred.

A preferred film layer for use in the present invention is described in EP 1720508A1 the description of which film layer is herein incorporated by reference. Such films when used in the present invention can have a vapor permeability of from about 50 to about 250 g/m², or optionally from about 100 to about 150 g/m² when measured over 24 hours using the Water Method (4.2) of the Water Vapor Transmission Test ASTM E96-00.

### Applicator

In order to avoid bending, wrinkling, or crumpling of the film layer 1, the applicator 2 is formed so as to have a bending stiffness greater than the bending stiffness of the film layer 1. The applicator 2 may be made from paper or cardboard material or from a metal, such aluminium or from a plastics material, such as polyester, such as polyethyleneterephthalate (PETP), which may optionally be coated (not shown in FIGures) with one or more coatings for providing desired properties, such as releasability (i.e. adhering properties) for achieving the desired releasability effects, cf. the above discussion. The adhering properties (or releasability) of the applicator 2 may thus vary in dependence of the choice of material for the coating. Suitable coating material groups may e.g. comprise silicone, metals, and Teflon™. It has been found that silicone materials are generally well suited for the coating, as various available silicone materials provide a useful variety of adhering properties. Once silicone materials have been identified as a suitable group of materials for the coating of the supporting surface of the carrier system, it is a matter of routine experimentation to identify that specific material, which is best suited for a particular embodiment or purpose.

Handling of the applicator 2 may, for certain uses, be facilitated if the bending stiffness, density and dimensions of the applicator 2 are such that essentially no deflection is caused to the applicator 2 by its own weight when the applicator 2 is supported at one end (or held by a finger at such one end). This is in particular useful in embodiments in which the applicator 2 extends directionally opposite and longitudinally from one end (or a first end) attached to the film layer 1, essentially in the plane of the film layer 1, forming an opposite end (second end) with middle portion between the opposite ends (i.e., first and second ends) as illustrated in FIGS. 2-5 and 7.

### Release Liner

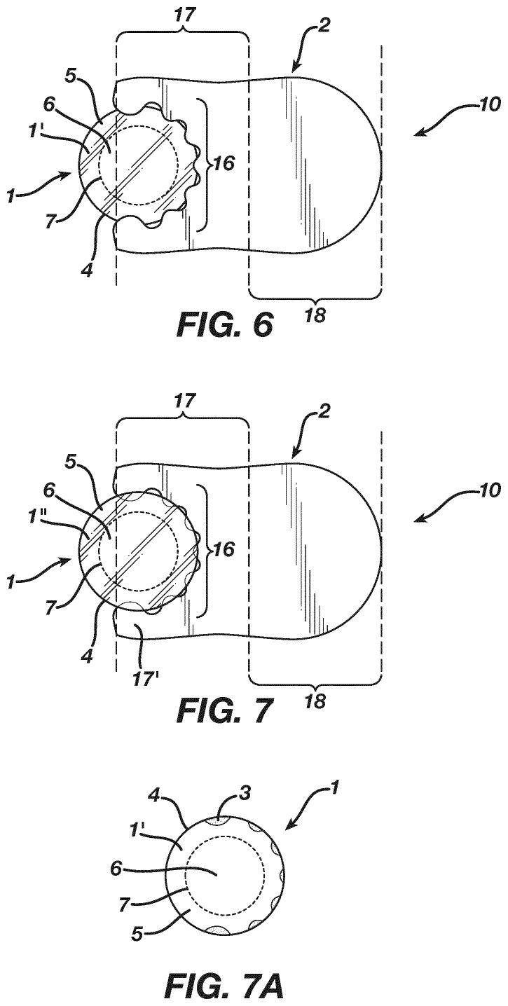

In certain embodiments, the film applicator assembly 10, optionally, further comprises a release liner 8. The term “release liner” and variants thereof refer to a web of material that has at least one surface arranged and configured for application to the top surface 1′ (and/or, optionally, the bottom surface 1″, if desired or necessary) and be removable from such surface prior to application despite the adhesive properties top surface 1′ (or, in some embodiments, the adhesive properties of both the top and bottom surfaces 1′ and 1″). In certain embodiments, the release liner 8 is arranged and configured such that release liner 8 covers at least a portion of the top surface 1′ which is not covered by the applicator 2, preferably release liner 8 covers all the top surface 1′ which is not covered by the applicator 2. In certain embodiments, the release liner 8 is shaped to align with cutout periphery 16 so as to enable release liner 8 to fit into and abut against cutout periphery 16 (as illustrated in FIG. 6).

Generally, the release liner 8 is used to isolate the adhesive surfaces 1′ or/and 1″ of the film layer 1 from the environment so as to retain the surface tack thereof prior to use. The release liner 8 is removed to expose the pressure sensitive adhesive for use. In certain embodiments, the release liner 8 is fixed attached to a separate sheet material 9, which sheet material 9 is sized so as to extend beyond at least one edge of the release liner 8 so as to form a tab 11. The tab 11 is formed such that when tab 11 is pulled, it causes the affixed release liner to pull away, with the sheet material 9 from the top and bottom surfaces 1′ and 1″ despite any adhesive properties associated with such surfaces.

As used herein the specification and the claims, the term “release agent” and variants thereof relate to a coating or other surface treatment to enhance the removal of release liner 8 from an adhesive surface in contact therewith. One of ordinary skill in the art will recognize appropriate release agents, including without limitation, coatings such as silicone-based (including siloxane) coatings, polyvinyl octadecyl carbonate-based coatings (PVODC), and other surface treatments.

The film applicator assembly 10 of the present invention may, optionally, also further comprise a protective paper layer 12 may optionally be used to prevent leakage or transfer of the film adhesive (e.g., hydrocolloid) onto the primary packaging (printed wrapper), which might cause film layer to stick to primary packaging. The protective paper layer 12 may be a siliconized paper, a nonwoven or any other releasable material as is known in the art.

As illustrated in FIG. 7, film layer 1, as described herein, is obtained and an applicator 2, as described herein, is releasably adhesively connected to the top surface 1′ of the film layer 1. As further illustrated, portions of top surface 1′ of the film layer 1 which remain exposed after connection of applicator 2 can be, optionally, covered and isolated from the external environment by release liner 8 (optionally affixed to sheet material 9 to form tab 11). Also illustrated are, optional, use of protective layer 12 and, optional, incorporation of film applicator assembly 10 into wrapper packaging formed by sealing of top wrapper packaging layer 13 to bottom wrapper packaging layer 14.

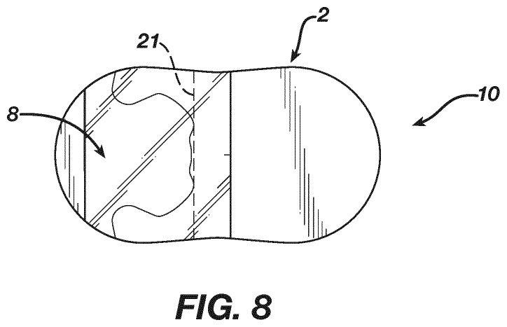

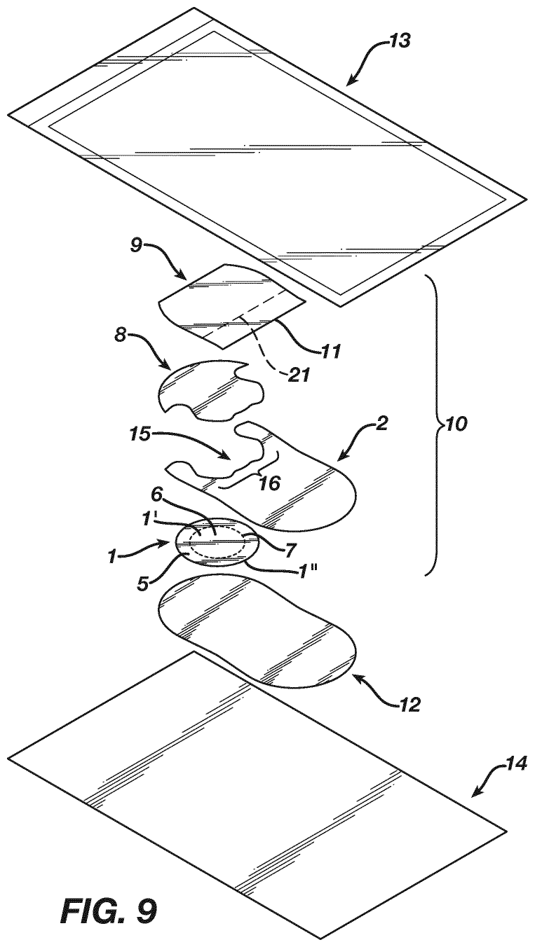

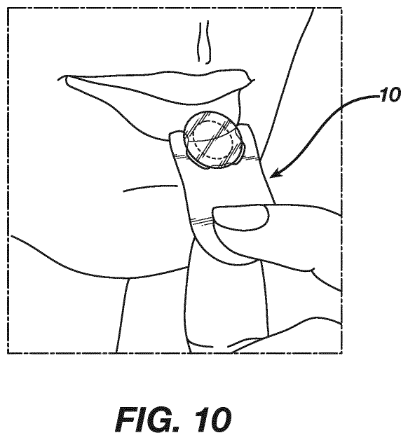

FIG. 8 illustrates a method of using the film applicator assembly 10 of the present invention. If present, release liner 8 is first removed from the top surface 1′ of film layer 1 prior to application by the user. The top surface 1′ of film layer 1 is then applied to the site of the sore or wound as shown in FIG. 8. Once the top surface 1′ of film layer 1 is adhered to the site of the sore or wound, the applicator 2 is pulled away from the film, preferably in the direction of the surface to which film layer 1 is adhered.

The general process of manufacturing the film applicator assembly 10 described above may be any of those conventionally known to produce films and film applicators. The applicator 2 and film layer 1 can be obtained by any methods available at present. For example, an extrusion process may be used for obtaining the applicator 2 and cut to desired shape. In the same way, the film layer 1 can be made in any known manner.

The film applicator assembly 10 described above may also be ideally suited to deliver a film layer 1 further comprising one or more active ingredients such as therapeutics to the surface of the skin. Illustrative classes of active ingredients that may be delivered to the skin via film layer 1 include, but are not limited to, antibiotics, analgesics, antipyretics, antimicrobials, antiseptics, antiallergics, anti-acne, anesthetics, anti-inflammatories, hemostats, cosmetics, vitamins, vasodilators, emollients, pH regulators, antipruritics, counterirritants, antihistamines and steroids. Specific active ingredients that may be delivered to the skin via the dressings of the invention include chlorhexidine, neomycin sulfate, polymyxin-B sulfate, zinc bacitracin, benzalkonium chloride, cetylpyridinium chloride, bupivacaine, tetracaine, cincaine, lidocaine, benzocaine, silver sulfadiazine, hydrocortisone, metandienone, trypsin, tolazoline, heparin, pramoxine, aloe vera, tretinoin, retinol, retinaldehyde, menthol, capsaicin, alpha hydroxy acids and vitamins such as Vitamin E.

Due to its discrete appearance and the easy applicability, the film layer 1 of the invention may be used for the treatment of herpes, and may be advantageously used with active ingredients known per se for such purposes being contained in the adhesive layer or being applied thereto. Suitable antiviral active ingredients for the treatment of herpes may for example comprise aciclovir or penciclovir.

The above mentioned pharmaceutically active ingredients may be applied to the top surface 1′ of film layer 1, or they may be mixed into the adhesive layer prior to addition of the backing layer.

In one embodiment of the invention, a gel or cream containing the active ingredients may be applied to the patch top surface 1′ of film layer 1 before application to human or animal surfaces. An amount of the gel or cream may be applied to the central part of the film layer 1 before application to the treatment site. In the treatment of Herpes it may be advantageous to apply an Acyclovir containing cream or gel such a Zovir before application to the Herpes site.

The top surface 1′ of film layer 1 may comprise one or more cavities for accommodating active ingredients. The cavities may be in the form of a dome shaped portion or an indentation.

The specification and embodiments above are presented to aid in the complete and non-limiting understanding of the invention disclosed herein. Since many variations and embodiments of the invention can be made without departing from its spirit and scope, the invention resides in the claims hereinafter appended.

The invention comprises the following items:
1. A film applicator assembly comprising:
c) a film layer having a top surface having adhesive properties, the top surface having a surface area; and
d) an applicator releasably adhesively connected to the top surface, contacting at least two contact regions of the surface to which it is releasably affixed;
wherein the total surface area of the contact regions contacted by the applicator is less than about 40% of the total surface area of the surface to which the applicator is releasably adhesively connected.
2. The film applicator assembly of item 1, wherein the film layer comprises an adhesive material in an amount sufficient to provide adhesive properties.
3. The film applicator assembly of item 1, wherein the adhesive properties are provided by an adhesive film coating.
4. The film applicator assembly of item 1, wherein the applicator contacts at least three discrete regions of the surface area of the surface to which it is releasably adhesively connected.
5. The film applicator assembly of item 4, wherein the total surface area of the discrete regions contacted by the applicator is less than about 30% of the total surface area of the surface to which the applicator is releasably adhesively connected.
6. The film applicator assembly of item 5, wherein the total surface area of the discrete regions contacted by the applicator is less than about 20% of the total surface area of the surface to which the applicator is releasably adhesively connected.
7. The film applicator assembly of item 1, further comprising a release liner releasably adhesively connected to the first surface of the film layer and contacting at least a portion of the surface area of the first surface not in contact with the applicator.
8. The film applicator assembly of item 1, further comprising a protective liner having a top and bottom surface, protective liner positioned adjacent to the applicator and film layer such that the second surface of the film layer contacts the top surface of the protective liner.
9. A film applicator assembly comprising:
a) a film layer having a top surface having adhesive properties, the top surface having:
   i. an outer edge defining a perimeter
   ii. a surface area, the surface area having a peripheral surface area and an internal surface area, the peripheral surface area extending from the perimeter to the internal surface area; and
b) an applicator releasably adhesively connected to the top surface at one or more contact regions within the peripheral surface area wherein the contact regions do not extend within the internal surface area.
10. The film applicator assembly of item 9, wherein the applicator is releasably adhesively connected to the top surface at one continuous contact region.
11. The film applicator assembly of item 9, wherein the applicator is releasably adhesively connected to the top surface at two or more discontinuous contact regions.
12. A film applicator assembly comprising:
a) an applicator having a gripping end and an applicator end opposite the gripping end, the applicator end having a surface and a cutout formed therein; and
b) a film layer defining a top surface and one or more outer edges, the top surface having a surface area, the top surface of the film layer being in releasable adhesive contact with the surface of the applicator end and overlaying the cutout such that less than 40% of the surface area of the top surface of the film layer is in releasable adhesive contact with the applicator end.
13. The film applicator assembly of item 12, wherein from about 40% to about 1% of the surface area of the top surface of the film layer is in releasable adhesive contact with the applicator end.
14. The film applicator assembly of item 13, wherein from about 30% to about 2% of the surface area of the top surface of the film layer is in releasable adhesive contact with the applicator end.
15. The film applicator assembly of item 14, wherein from about 20% to about 3% of the surface area of the top surface of the film layer is in releasable adhesive contact with the applicator end.
16. The film applicator assembly of item 12, wherein the outer edge of the film layer is in the form of a circular edge.
17. The film applicator assembly of item 12, wherein the top surface of film layer releasably adhesively contacts the surface of the applicator end at least two contact regions.
18. The film applicator assembly of item 12, wherein the cutout is positioned at the applicator end such that the cutout forms an open cutout.
19. The film applicator assembly of item 12, wherein the cutout is positioned at the applicator end such that the cutout forms a closed cutout.

1. A film applicator assembly comprising:
a) a film layer having a top surface having adhesive properties and one or more outer edges, the top surface having a surface area; and
b) an applicator releasably adhesively connected to the top surface, contacting at least one contact region of the top surface to which it is releasably affixed;
wherein the total surface area of the contact regions contacted by the applicator is less than about 40% of the total surface area of the surface to which the applicator is releasably adhesively connected.

2. The film applicator assembly of claim 1, wherein the top surface of the film layer comprises
i. an outer edge defining a perimeter;
ii. a surface area, the surface area having a peripheral surface area and an internal surface area, the peripheral surface area extending from the perimeter to the internal surface area; and
wherein the contact regions extend within the peripheral surface area and do not extend within the internal surface area.

3. The film applicator assembly of claim 1, wherein the applicator has a gripping end and an applicator end opposite the gripping end, the applicator end having a surface and a cutout formed therein, wherein the top surface of the film layer overlays the cutout.

4. The film applicator assembly of claim 3, wherein from about 40% to about 1% of the surface area of the top surface of the film layer is in releasable adhesive contact with the applicator end.

5. The film applicator assembly of claim 1, wherein the film layer comprises an adhesive material in an amount sufficient to provide adhesive properties and/or wherein the adhesive properties are provided by an adhesive film coating.

6. The film applicator assembly of claim 1, wherein the applicator contacts one continuous contact region of the surface area of the top surface to which it is releasably adhesively connected.

7. The film applicator assembly of claim 1, wherein the applicator contacts two or more discontinuous contact regions of the surface area of the top surface to which it is releasably adhesively connected.

8. The film applicator assembly of claim 1, wherein the applicator contacts at least three discrete regions of the surface area of the top surface to which it is releasably adhesively connected.

9. The film applicator assembly of claim 8, wherein the total surface area of the discrete regions contacted by the applicator is less than about 30% of the total surface area of the surface to which the applicator is releasably adhesively connected.

10. The film applicator assembly of claim 1, further comprising a release liner releasably adhesively connected to the top surface of the film layer and contacting at least a portion of the surface area of the top surface not in contact with the applicator.

11. The film applicator assembly of claim 10, wherein the release liner covers all the top surface of the film layer which is not covered by the applicator.

12. The film applicator assembly of claim 10, wherein the release liner is a protective liner having a top and bottom surface, the protective liner being positioned adjacent to the applicator and film layer such that the second surface of the film layer contacts the top surface of the protective liner.

13. The film applicator assembly of claim 2, wherein the outer edge of the film layer is in the form of a circular edge.

14. The film applicator assembly of claim 3, wherein the cutout of the applicator is positioned at the applicator end such that the cutout forms an open cutout.

15. The film applicator assembly of claim 3, wherein the cutout of the applicator is positioned at the applicator end such that the cutout forms a closed cutout.

16. The film applicator assembly of claim 1, wherein the film layer has a total thickness of 100-200 μm.

17. (canceled)

18. The film applicator assembly of claim 1, wherein the surface area of the top surface of film layer is in the range of 0.12 to 5 cm².

19. The film applicator assembly of claim 1, wherein the film layer is for facial application.

20. The film applicator assembly of claim 1, wherein the top surface formed by an adhesive layer is uninterrupted.

21. The film applicator assembly of claim 1, wherein the film layer comprises one or more active ingredients selected from the group consisting of antibiotics, analgesics, antipyretics, antimicrobials, antiseptics, antiallergic s, anti-acne, anesthetics, anti-inflammatories, hemostats, cosmetics, vitamins, vasodilators, emollients, pH regulators, antipruritics, counterirritants, antihistamines and steroids.

22. (canceled)
